(19) United States
(12) Patent Application Publication
(10) Pub. No.: US 2025/0295719 A1
(43) Pub. Date: Sep. 25, 2025

(54) IL-27 EXPRESSING ONCOLYTIC VIRUSES

(71) Applicant: THE RESEARCH INSTITUTE AT NATIONWIDE CHILDREN'S HOSPITAL, COLUMBUS, OH (US)

(72) Inventor: Kevin A. CASSADY, Columbus, OH (US)

(21) Appl. No.: 18/869,720

(22) PCT Filed: May 31, 2023
(86) PCT No.: PCT/US2023/023966

§ 371 Date: Nov. 26, 2024

Related U.S. Application Data
(60) Provisional application No. 63/347,246, filed on May 31, 2022.

(51) Int. Cl. A61K 35/763; A61P 35/00; C07K 14/54; C12N 7/00

(52) U.S. Cl. CPC A61K 35/763; A61P 35/00; C07K 14/54; C12N 7/00; C12N 2710/16621; C12N 2710/16632

(57) ABSTRACT

A recombinant interleukin-27 (IL27) expressing virus is described. The recombinant IL27 expressing virus comprises an oncolytic virus comprising one or more exogenous nucleic acid sequences capable of expressing in IL27 protein or a biologically active portion thereof, the exogenous nucleic acid sequences being operably linked to an expression control sequence. Methods of treating cancer by in a subject by contacting a cancer cell of the subject with a recombinant IL27 expressing virus are also described.

### CROSS-REFERENCE TO RELATED APPLICATIONS

This application claims priority to U.S. Provisional Patent Application No. 63/347,246, filed on May 31, 2022, which is hereby incorporated by reference in its entirety.

### GOVERNMENT SUPPORT

This invention was made with government support under Grant No. CA232561 awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety.

### BACKGROUND

IL27 is a cytokine within the IL12 family that has been neglected because it was thought to increase Treg activity and promote tumor growth. Cytokines in the IL12 family require 2 subunits for activity, and these subunits are shared between related but different IL12 family members (e.g., IL27, IL30, and IL35). Vignali D., & Kuchroo, V., Nat. Immunol. 13(8), 722-728 (2012). Cytokines such as IL-12 have been used to directed an anti-tumor immune response, but the short half-life of these cytokines when administered as proteins have diminished their effectiveness.

Rapid increases in molecular biotechnology techniques provided means to develop novel strategies to harness the immune system for cancer therapy. Currently, a number of approaches, including adoptive cell therapies, monoclonal antibodies, checkpoint inhibitors, and oncolytic viruses constitute the most prominent advancements in cancer treatment due to the capacity to provide durable and effective clinical responses in cancer patients. Lizee et al., Annu Rev Med., 64:71-90 (2013).

Oncolytic viruses are genetically engineered or naturally occurring viruses that selectively replicate in and kill cancer cells without harming normal tissues. In gliomas, several kinds of conditionally replication competent viruses have been shown to be useful in animal models. For all of these oncolytic viruses, the goal is the intratumoral spread of the virus and the ability to selectively kill cancer cells. A number of oncolytic virus drugs for cancer treatment have been recently approved. Fukuhara et al., Cancer Sci. 107, 1373-1379 (2016). Oncolytic herpes simplex viruses (HSVs) were initially designed and constructed for the treatment of brain tumors, and their safety has been extensively tested in mice and primates, which are extremely sensitive to HSV. However, there remains a need for means of treating cancer with oncolytic viruses capable of anti-tumor immune activation.

### SUMMARY

The inventors postulated that some of the tumor promoting activities previously associated with IL27 were likely related to subunits shared with other IL12 family cytokines or they represented natural negative feedback mechanisms to reduce the immune response following IL27 expression. The inventors hypothesized that IL27 expressed from an oncolytic Herpes Simplex Virus (HSV) would improve T-cell and cytotoxic activity and improve survival in tumor-bearing animals. Data supporting this hypothesis came from early clinical trial analyses by the inventors. They then assessed patients with improved survival after oncolytic HSV treatment and determined that genes associated with IL27 expressing oncolytic virus directly correlated with improved patient survival. Based on this, they created IL27-expressing oncolytic viruses to further test the hypothesis that IL27 expression by oncolytic viruses improved survival and led to immune clearance of tumors.

A major advantage of IL27 over current IL12-based cytokine approaches is that IL27 exhibits both antitumor and anti-inflammatory activity. Current cytokine-based immunotherapies often produce dose-limiting and dangerous cytokine side effects which limit their success. IL27 not only improves T cell Th1 function, but also has anti-inflammatory properties.

Accordingly, in one aspect, the present invention provides a recombinant interleukin-27 (IL27) expressing virus. The recombinant IL27 expressing virus comprises an oncolytic virus comprising one or more exogenous nucleic acid sequences capable of expressing an IL27 protein or a biologically active portion thereof, which is operably linked to an expression control sequence.

In some embodiments, the oncolytic virus includes a deletion mutation that decreases the virulence of the oncolytic virus. In additional embodiments, the oncolytic virus is a herpesvirus, such as an α herpesvirus or an HSV-1 herpesvirus. In further embodiments, the virus is an HSV-1 herpesvirus, and modified to include a deletion of the herpesvirus gamma (1)34.5 gene (γ134.5) locus, while in yet further embodiments the oncolytic virus comprises a viral nucleic acid sequence encoding an RNA-dependent Protein Kinase R (PKR) evasion protein that does not cause virulence.

In additional embodiments, the exogenous nucleic acid sequences operably linked to an expression control sequence and capable of expressing in IL27 protein or a functional portion thereof are inserted to replace the deleted γ134.5 locus. In further embodiments, the exogenous nucleic acid sequences capable of expressing in IL27 protein are a bi-cistronic mIL27 gene inserted into the oncolytic herpesvirus at the deleted γ134.5 locus. In yet further embodiments, the IL27 protein comprises an amino acid sequence that is at least 95% identical to the wild-type human IL27 sequence.

Another aspect of the present invention provides a method of treating cancer by in a subject by contacting a cancer cell of the subject with a recombinant interleukin-27 (IL27) expressing virus. The recombinant virus comprises an oncolytic virus comprising one or more exogenous nucleic acid sequences operably linked to an expression control sequence and capable of expressing in IL27 protein or a biologically active portion thereof. In additional embodiments, the recombinant IL27 expressing virus is administered in a pharmaceutically acceptable carrier.

In some embodiments, the cancer is glioblastoma. In additional embodiments, the cancer cell is contacted ex vivo, while in other embodiments the cancer cell is contacted in vivo. In yet further embodiments, the method further comprises administering chemotherapy or radiation therapy to the subject.

In some embodiments, the oncolytic virus used in the method includes a deletion mutation that decreases the virulence of the oncolytic virus. In further embodiments, the oncolytic virus is a herpesvirus, while in yet further embodiments the herpesvirus is an HSV-1 herpesvirus.

### BRIEF DESCRIPTION OF THE FIGURES

FIG. 1 provides a schematic representation of cytokines of the IL12 family (specifically, IL12, IL23, IL27, IL35, and IL39) embedded within a membrane, and highlighting the two subunits of the cytokine required for activity.

FIG. 2 provides a heat map image and schematic representations of Ingenuity-based upstream regulatory analysis of the 502 significant genes correlating with G207-survival, which show that these genes map to Immune response pathways. These include both intrinsic antiviral response pathways (RED) and adaptive Immune Response Pathways (including antigen presentation: BLUE). Log₂ fold Change (best responder relative to worst responders). Gene expression and activation Z score is represented as a heatmap for the transcriptional regulators significantly associated with improved survival (Il27 highlighted with 6.61x gene upregulation, Z score=2.177, p=0.013). Schematic representation of the significant upstream responses.

FIGS. 3A and 3B provide a graph and schematic representation of C027 and anti-glioma activity: A) Schematic representation of C027, a C134-based oHSV encoding 2 copies of bi-cistronic miL27 gene within the ICP34.5 locus is shown. The inventors are constructing the other oHSV-based constructs (non-C134 based versions of the miL27 and hiL27 versions as well). B) C027 gliomas implanted in the flanks of immunocompetent C57/BI6 mice show that C027 cleared tumors and outperformed parent C135 and our other cytokine expression viruses C021 and C002.

FIG. 4 provides a graph showing data obtained in peripheral tumor tissue comparing the effects of C027, C134/154, and saline on probability of survival.

FIG. 5 provides a nucleotide sequence for the first 4056 nucleotides of mIL27 in the RL1 ICP-34-5 locus (SEQ ID NO: 1).

FIG. 6 provides a nucleotide sequence for the humanized IL27 RL1 viral sequence (SEQ ID NO: 2).

FIG. 7 provides a PNG image of humanized IL27 location in RL1 locus.

### DETAILED DESCRIPTION

The present invention provides a recombinant interleukin-27 (IL27) expressing virus. The recombinant IL27 expressing virus comprises an oncolytic virus comprising one or more exogenous nucleic acid sequences capable of expressing in IL27 protein or a biologically active portion thereof, the exogenous nucleic acid sequences being operably linked to an expression control sequence. Methods of treating cancer by in a subject by contacting a cancer cell of the subject with a recombinant IL27 expressing virus are also provided.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, and other references mentioned herein are incorporated by reference in their entirety.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein and in the appended claims, the singular forms “a”, “and”, and “the” include plural referents unless the context clearly dictates otherwise. Thus, for example, reference to “a sample” also includes a plurality of such samples and reference to “the splicing regulator protein” includes reference to one or more protein molecules, and so forth.

As used herein, the term “about” refers to +/−10% deviation from the basic value.

As used herein the term “nucleic acid” or “oligonucleotide” refers to multiple nucleotides (i.e. molecules comprising a sugar (e.g. ribose or deoxyribose) linked to a phosphate group and to an exchangeable organic base, which is either a substituted pyrimidine (e.g. cytosine (C), thymidine (T) or uracil (U)) or a substituted purine (e.g. adenine (A) or guanine (G)). The term shall also include polynucleosides (i.e. a polynucleotide minus the phosphate) and any other organic base containing polymer. Purines and pyrimidines include but are not limited to adenine, cytosine, guanine, thymidine, inosine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Other such modifications are well known to those of skill in the art. Thus, the term nucleic acid also encompasses nucleic acids with substitutions or modifications, such as in the bases and/or sugars.

The term “base” encompasses any of the known base analogs of DNA and RNA. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

“Exogenous,” as used herein with reference to a nucleic acid sequence, means that it is foreign to the cell into which the vector is being introduced or that the sequence is homologous to a sequence in the cell but in a position within the host cell nucleic acid in which the sequence is ordinarily not found.

The term “recombinant virus,” as used herein, defines a recombinant virus (e.g., an IL27 expressing virus) comprising: (a) the DNA of, or corresponding to, at least a portion of the genome of an oncolytic virus that is capable of transducing into a target cell at least one selected gene and is capable of promoting replication and packaging; and (b) at least one selected gene (or transgene) operatively linked to at least one regulatory sequence directing its expression, the gene flanked by the DNA of (a) and capable of expression in the target cell in vivo or in vitro. Thus, a “recombinant virus” (e.g., IL27 expressing virus) means a virus that has been genetically altered, e.g., by the addition or insertion of a selected gene (e.g., a gene encoding IL27).

A “gene,” or a “sequence which encodes” a particular protein, is a nucleic acid molecule which is transcribed (in the case of DNA) and translated (in the case of mRNA) into a polypeptide in vitro or in vivo when placed under the control of one or more appropriate regulatory sequences. A gene of interest can include, but is no way limited to, cDNA from eukaryotic mRNA, genomic DNA sequences from eukaryotic DNA, and even synthetic DNA sequences. A transcription termination sequence will usually be located 3′ to the gene sequence. Typically, a polyadenylation signal is provided to terminate transcription of genes inserted into a recombinant virus.

The term “operably linked,” as used herein, refers to the arrangement of various nucleic acid molecule elements relative to each other such that the elements are functionally connected and are able to interact with each other. Such elements may include, without limitation, a promoter, an enhancer, a polyadenylation sequence, one or more introns and/or exons, and a coding sequence of a gene of interest to be expressed. The nucleic acid sequence elements, when operably linked, can act together to modulate the activity of one another, and ultimately may affect the level of expression of the gene of interest, including any of those encoded by the sequences described above.

As used herein, the term “therapeutically effective amount” is intended to mean the amount of vector which exerts oncolytic activity, causing attenuation or inhibition of tumor cell proliferation, leading to tumor regression. An effective amount will vary, depending upon the pathology or condition to be treated, by the patient and his or her status, and other factors well known to those of skill in the art. Effective amounts are easily determined by those of skill in the art. In some embodiments a therapeutic range is from 10³ to 10¹² plaque forming units introduced once. In some embodiments a therapeutic dose in the aforementioned therapeutic range is administered at an interval from every day to every month via the intratumoral, intrathecal, convection-enhanced, intravenous or intra-arterial route.

Treat”, “treating”, and “treatment”, etc., as used herein, refer to any action providing a benefit to a patient at risk for or afflicted with a disease, including improvement in the condition through lessening or suppression of at least one symptom, delay in progression of the disease, prevention or delay in the onset of the disease, etc. Treatment also includes partial or total destruction of the undesirable proliferating cells with minimal destructive effects on normal cells. A subject at risk is a subject who has been determined to have an above-average risk that a subject will develop cancer, which can be determined, for example, through family history or the detection of genes causing a predisposition to developing cancer.

The term “subject,” as used herein, refers to a species of mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos. The term does not denote a particular age or sex. Thus, newborn subjects and infant subjects, as well as fetuses, whether male or female, are intended to be included within the scope of this term.

Unless defined otherwise, all technical and scientific terms used herein have the same meaning as commonly understood by one of ordinary skill in the art to which this invention belongs.

### IL-27 Expressing Oncolytic Viruses

In one aspect, the invention provides a recombinant interleukin-27 (IL27) expressing virus, comprising: An oncolytic virus comprising one or more exogenous nucleic acid sequences capable of expressing in IL27 protein or a biologically active portion thereof, the exogenous nucleic acid sequences being operably linked to an expression control sequence.

An oncolytic virus is a virus that has oncolytic activity. Oncolytic activity refers to cytotoxic effects in vitro and/or in vivo exerted on tumor cells without any appreciable or significant deleterious effects to normal cells under the same conditions. The cytotoxic effects under in vitro conditions are detected by various means as known in prior art, for example, by staining with a selective stain for dead cells, by inhibition of DNA synthesis, or by apoptosis. Detection of the cytotoxic effects under in vivo conditions is performed by methods known in the art.

The recombinant IL27 expressing virus includes an oncolytic virus. Examples of oncolytic viruses include Herpesvirus, Varicella Zoster virus, Vaccinia virus (e.g., vaccinia virus vTF7-3), Newcastle Disease virus, Adenovirus, Poxvirus, Picornavirus, Reovirus, and Sindbis virus. For a review of oncolytic viruses in cancer treatment, see Cook, M., and Chauhan, A., Int J Mol Sci., 21(20): 7505 (2020). In some embodiments, the oncolytic virus is selected from Herpesvirus, Varicella Zoster virus, and Vaccinia virus.

In some embodiments, the oncolytic viruses is a Herpesvirus. Genetically modified herpesvirus are attractive as oncolytic vectors for a number of reasons: 1) procedures for constructing recombinant herpesvirus are well established; 2) multiple genes can be deleted and/or replaced with therapeutic foreign genes without affecting the replication capacity of the virus; 3) considerable experience with the biology of herpesvirus and its behavior in humans and nonhuman primates exists in the literature; and 4) modified herpesviruses can be engineered to retain sensitivity to standard antiviral drug therapy as a “built-in” safety feature. Furthermore, the genome size of the Herpes Simplex Virus, 152 kb, allows transfer of genes 30 kb or more in size.

There are more than 120 animal herpesviruses. All herpesviruses are divided into three subsets: the alpha (α), beta (β) and gamma (γ) herpesviruses. There are 8 human herpesviruses, which are split between the three subsets. Alpha Herpesviruses include Herpes Simplex Virus 1 (HSV-1), HSV-2, and Varicella Zoster Virus (VZV). Beta Herpesviruses include Human Cytomegalovirus (HCMV), Human Herpesvirus 6 (HHV-6), and Human Herpesvirus 7 (HHV-7). Gamma Herpesvirus include Epstein Barr Virus (EBV) and Gamma Kaposi's Sarcoma Herpesvirus. Accordingly, in some embodiments the herpesvirus included in the IL27 expressing oncolytic virus is an α herpesvirus, while in further embodiments the herpesvirus included in the IL27 expressing oncolytic virus is an HSV-1 herpesvirus.

HSV-1-based oncolytic viruses are particularly preferred because of: (1) their ability to infect a wide variety of tumors; (2) their inherent cytolytic nature; (3) their well-characterized large genome (152 Kb) that provides ample opportunity for genetic manipulations wherein many of the non-essential genes can be replaced by therapeutic genes; (4) their ability to remain as episomes that avoid insertional mutagenesis in infected cells; and (5) the availability of anti-herpetic drugs to keep in check possible undesirable replication.

In some embodiments, the oncolytic virus includes a deletion mutation that decreases the virulence of the oncolytic virus. Examples of modified HSV-1 vectors that target malignant glioma include two deletion mutant genes, ICP6 (U_{L}39 gene product), the large subunit of HSV-1 ribonucleotide reductase (RR), and ICP34.5 (34.5 gene product), a multifunctional protein that is also related to neurovirulence. While the lack of ICP6 restricts virus replication to non-dividing cells but allows replication to continue in cells with defects in the p16 tumor suppressor pathway, deletions of both 34.5 genes suppresses HSV-1 encephalitis. Accordingly, in some embodiments, the virus is an HSV-1 herpesvirus that has been modified to include a deletion of the herpesvirus gamma (1)34.5 gene (γ134.5) locus.

Many viruses evolved to counteract PKR activity by using their own viral products or by hijacking cellular proteins, acting at the different steps in the cascade of PKR activation. Accordingly, in some embodiments, the oncolytic virus includes a viral nucleic acid sequence encoding a PKR evasion protein that does not cause virulence. The RNA-dependent Protein Kinase R (PKR) is a pattern recognition receptor that is a key component of an innate immune system, and recognizes imperfectly double-stranded non-coding viral RNA molecules via its N-terminal double-stranded RNA binding motifs. PKR is also closely linked to p53. See Cesaro T. and Michiels, T., Front Microbiol, 12:757238 (2021). For example, the viral product ICP34.5 acts as a PP1 regulator subunit in HSV-1. He et al., J. Biol. Chem. 273, 20737-20743 (1998). Additional PKR evasion proteins for HSV-1 include Us11 (Cassady K. and Gross M., J. Virol. 76, 2029-2035 (2002)) and VHS (Dauber et al., J. Virol. 85, 5363-5373 (2011)).

In some embodiments, the exogenous nucleic acid sequences operably linked to an expression control sequence and capable of expressing in IL27 protein or a functional portion thereof are inserted in the oncolytic virus to replace the deleted γ134.5 locus. By inserting the immune active gene(s) comprising IL27 into the same locus as a loss of function mutation (i.e., deleting the principal neurovirulence gene g134.5), a virus is generated that is safe and avoids genetic modification to other viral transcripts In further embodiments, the exogenous nucleic acid sequences capable of expressing in IL27 protein are a bi-cistronic mIL27 gene, which can also be inserted to replace the deleted γ134.5 locus.

In some embodiments, the herpesvirus is modified to include a deletion of the herpesvirus gamma (1)34.5 gene (γ₁34.5) or a nucleic acid with at least about 70% homology to the γ₁34.5 gene. The modification to the herpesvirus nucleic acid sequence can also be a modification of a nucleic acid with at least about 70-99% homology, including 70%, 75%, 80%, 85%, 90%, or 95% homology, to the γ₁34.5 gene.

Modifications that can be made to the γ₁34.5 gene include one or more mutations, deletions, insertions and substitutions. Thus, the modification to the herpesvirus nucleic acid sequence can comprise the complete or partial deletion of the γ₁34.5 gene from HSV-1. The modification can comprise an inserted exogenous stop codon or other nucleotide or nucleotides. The modification can comprise the mutation or deletion of the promoter or the insertion of an exogenous promoter that alters expression of the γ₁34.5 gene. The modification can comprise one or more inserted nucleotides that results in a codon frame-shift. Furthermore, the second viral nucleic acid sequence of the chimera could be substituted for the γ₁34.5 gene. Methods for making the modifications described herein are well known to those skilled in the art and are described in more detail below.

In general, it is understood that one way to define any known variants and derivatives of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman, Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by, for example, the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989, which are herein incorporated by reference for at least the material related to nucleic acid alignment. It is understood that any of the methods typically can be used and that, in certain instances, the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology. as defined herein, to a second sequence if the first sequence is calculated to have 80 percent homology to the second sequence using both the Zuker calculation method and the Pearson and Lipman calculation method, even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of the calculation methods, although, in practice, the different calculation methods will often result in different calculated homology percentages.

The disclosed nucleic acids may contain, for example, nucleotide analogs or nucleotide substitutes. Non-limiting examples of these and other molecules are discussed herein. It is understood that, for example, when a vector is expressed in a cell, the expressed mRNA will typically be made up of A, C, G, and U. A nucleotide analog is a nucleotide which contains some type of modification of either the base, sugar, or phosphate moieties. Modifications to nucleotides are well known in the art and include for example, 5-methylcytosine (5-me-C), 5-hydroxymethyl cytosine, xanthine, hypoxanthine, and 2-aminoadenine.

### Interleukin 27

The recombinant IL27 expressing virus includes exogenous nucleic acid sequences capable of expressing an IL27 protein or a biologically active portion thereof. In some embodiments, the exogenous nucleic acid sequences are inserted into an oncolytic herpesvirus at the γ₁34.5 locus. In further embodiments, the nucleic acid sequences capable of expressing in IL27 protein are a bi-cistronic mIL27 gene inserted into the oncolytic herpesvirus at the γ₁34.5 locus.

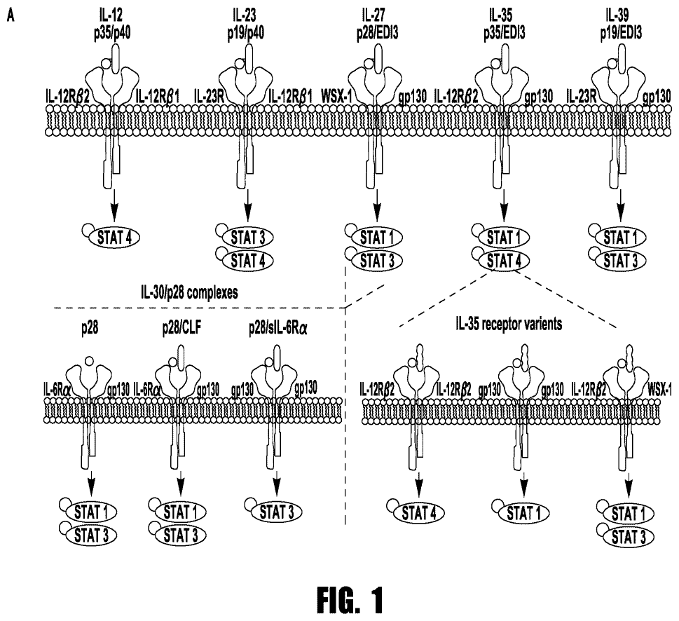

IL-27 is a cytokine within the IL12 family that has been less popular as a cancer therapeutic due to early observational studies that linked it to increase Treg activity, and being closely related to cytokines that promoter tumor growth. IL-27 is a heterodimeric cytokine consisting of a four-helix bundle, IL-27p28 (p28), with similarity to IL-6, complexed with a secreted binding protein, Epstein-Barr Virus-Induced 3 (Ebi3), with homology to type I cytokine receptors. Pflanz et al., J Immunol., 172(4):2225-31 (2004). Cytokines in the IL12 family require 2 subunits for activity, and these subunits are shared between related but different IL12 family members (e.g., IL27, IL30, and IL35). See FIG. 1. In some embodiments, the IL27 is human IL27, while in other embodiments the IL27 is murine IL27.

The recombinant IL27 expressing virus includes exogenous nucleic acid sequences capable of expressing an IL27 protein or a biologically active portion thereof. In some embodiments, the IL27 protein comprises an amino acid sequence that is at least 90% identical to the wild-type human IL27 sequence. In further embodiments, the IL27 protein comprises an amino acid sequence that is at least 95% identical to the wild-type human IL27 sequence. In some embodiments, the sequences include only conservative replacements of amino acids. In yet further embodiments, the IL27 protein comprises an amino acid sequence that is identical to the wild-type human IL27 alpha subunit sequence (SEQ ID NO: 3).

(SEQ ID NO: 3)
MGQTAGDLGWRLSLLLLPLLLVQAGVWGFPRPPGRPQLSLQELRREFTV
SLHLARKLLSEVRGQAHRFAESHLPGVNLYLLPLGEQLPDVSLTFQAWR
RLSDPERLCFISTTLQPFHALLGGLGTQGRWTNMERMQLWAMRLDLRDL
QRHLRFQVLAAGFNLPEEEEEEEEEEEEERKGLLPGALGSALQGPAQVS
WPQLLSTYRLLHSLELVLSRAVRELLLLSKAGHSVWPLGFPTLSPQP.

Examples of nucleotide sequences capable of expressing IL27 include the first 4056 nucleotides of mIL27 in the RL1 ICP-34-5 locus (SEQ ID NO: 1) and the nucleotide sequence for the humanized IL27 RL1 viral sequence (SEQ ID NO: 2).

A “biologically active” portion of a molecule, as used herein, refers to a portion of a larger molecule that can perform a similar function as the larger molecule. Merely by way of non-limiting example, a biologically active portion of a promoter is any portion of a promoter that retains the ability to influence gene expression, even if only slightly. Similarly, a biologically active portion of a protein is any portion of a protein which retains the ability to perform one or more biological functions of the full-length protein (e.g., binding with another molecule, phosphorylation, etc.), even if only slightly.

### Viral Vectors

The present invention provides an oncolytic virus comprising one or more exogenous nucleic acid sequences capable of expressing in IL27 protein or a biologically active portion thereof, the exogenous nucleic acid sequences being operably linked to an expression control sequence. The exogenous nucleic acids typically are included operably linked to an expression controlling system (i.e., an expression control sequence). For example, the inserted genes in a recombinant viral sequence usually contain promoters and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

The term “promoter,” as used herein, refers to a nucleic acid sequence that regulates, either directly or indirectly, the transcription of a corresponding nucleic acid coding sequence to which it is operably linked. The promoter may function alone to regulate transcription, or. in some cases, may act in concert with one or more other regulatory sequences such as an enhancer or silencer to regulate transcription of the gene of interest. The promoter comprises a DNA regulatory sequence, wherein the regulatory sequence is derived from a gene, which is capable of binding RNA polymerase and initiating transcription of a downstream (3′-direction) coding sequence. A promoter generally comprises a sequence that functions to position the start site for RNA synthesis. The best-known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one can position the 5′ end of the transcription initiation site of the transcriptional reading frame “downstream” of (i.e., 3′ of) the chosen promoter. The “upstream” promoter stimulates transcription of the DNA and promotes expression of the encoded RNA.

The promoter region can act as a constitutive promoter to maximize expression of the region of the transcription unit to be transcribed. In certain constructs, the promoter region can be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR. It has been shown that all specific regulatory elements can be cloned and used to construct expression vectors that are selectively expressed in specific cell types such as melanoma cells. For example, the glial fibrillary acidic protein (GFAP) promoter has been used to selectively express genes in cells of glial origin. Such tumor specific promoters can also be incorporated into the IL27 expressing viruses as well as the viral vectors described herein.

The spacing between promoter elements frequently is flexible, so that promoter function is preserved when elements are inverted or moved relative to one another. Depending on the promoter used, individual elements can function either cooperatively or independently to activate transcription.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages may be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the beta-lactamase (penicillinase), lactose and tryptophan (trp) promoter systems. As demonstrated herein, in some embodiments, a nestin promoter is used to drive expression of the gene of interest. In addition to producing nucleic acid sequences of promoters and enhancers synthetically, sequences may be produced using recombinant cloning and/or nucleic acid amplification technology, in connection with the recombinant virus disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 base pairs (bp) in length. Enhancers are cis-acting regulatory sequence involved in the transcriptional activation of a nucleic acid sequence, such as those for the genes, or portions or functional equivalents thereof. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples include, but are not limited to, the SV40 enhancer, the cytomegalovirus early promoter enhancer, the polyoma enhancer, and adenovirus enhancers.

Expression vectors used in viral vectors may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs are well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences, alone or in combination with the above sequences, to improve expression from, or stability of, the construct.

The viral vectors can also include a nucleic acid sequence encoding a marker product. This marker product is used to determine if the gene has been delivered to the cell and once delivered is being expressed. Marker genes include, for example, the E. coli lacZ gene, which encodes β-galactosidase, and green fluorescent protein (GFP). Markers can also be used in imaging techniques. Thus, a IL27 expressing vector that encodes a marker could be used to visualize a cancer cell or tumor. The size of the marked region or the intensity of the marker can be used to evaluate the progression, regression, or cure of cancer, for example.

Viral recovery and immunohistochemistry have been used successfully to monitor viral replication and spread in vivo. Bioluminescent and fluorescent protein expression by the virus can also be used to indirectly monitor viral replication and spread in the tumor. Genes encoding fluorescent reporter proteins (d2EGFP and dsRED monomer) or bioluminescent markers (firefly luciferase) are commonly used in recombinant viruses. Not only do these facilitate the screening and selection of recombinant viruses in vitro. The reporter genes also allow indirect monitoring of viral activity in in vivo studies.

### Methods of Making

The compositions disclosed herein and the compositions necessary to perform the disclosed methods can be made using any method known to those of skill in the art for that particular reagent or compound unless otherwise specifically noted. For example, the nucleic acids can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

The recombinant IL27 expressing viruses and viral vectors can also be made recombinantly as set forth in the examples or by other methods of making recombinant viruses as described in many standard laboratory manuals, such as Davis et al., Basic Methods in Molecular Biology (1986) and Sambrook et al., Molecular Cloning: A Laboratory Manual, 2nd Ed., Cold Spring Harbor Laboratory Press, Cold Spring Harbor, N.Y. (1989). Similar methods are used to introduce a gene of interest in methods of making the viral vector described herein. For example, recombinant viruses can be constructed using homologous recombination after DNA co-transfection. In this example, cells can be co-transfected with at least two different viruses containing the genes of interest and progeny virus plaque can be purified based upon loss of marker expression. Final verification of the correct genetic organization of candidate viruses can be verified by DNA hybridization studies using probes to the nucleic acids as described herein.

Specifically, a targeting plasmid can be constructed encoding the bicistronic IL27 subunits linked by a 2A ribosomal slippage element. The linearized plasmid is then introduced into RSCs with PacI digested C154 DNA and viral progeny selected using loss of EGFP expression and gain of IL27 expression from serial plaque selection on vero cells. The recombinant HSV can constructed using homologous recombination and the Pac I method. See Parker et al., Mol Pharm., 8(1): 44-49 (2011); the disclosure of which is incorporated herein by reference.

When the nucleic acid sequences are used recombinantly, the nucleic acid sequence may include the coding sequence for the mature polypeptide, by itself, or the coding sequence for the mature polypeptide in reading frame with other coding sequences, such as those encoding a leader or secretory sequence, a pre-, or pro- or prepro-protein sequence, or other fusion peptide portions. The nucleic acid sequence may also contain non-coding 5′ and 3′ sequences, such as transcribed, non-translated sequences, splicing and polyadenylation signals, ribosome binding sites and sequences that stabilize mRNA.

The nucleic acids may be used as hybridization probes for cDNA and genomic DNA or as primers for a nucleic acid amplification (PCR) reaction, to isolate full-length cDNAs and genomic clones encoding polypeptides and to isolate cDNA and genomic clones of other genes (including genes encoding homologs and orthologs from different species) that have a high sequence similarity.

The nucleic acids described herein, including homologs and orthologs from species, may be obtained by a process which comprises the steps of screening an appropriate library (as understood by one of ordinary skill in the art) under stringent hybridization conditions with a labeled probe or a fragment thereof; and isolating full-length cDNA and genomic clones containing said polynucleotide sequence. Such hybridization techniques are well known to the skilled artisan.

### Cancer Treatment

Another aspect of the invention provides a method of treating cancer by in a subject by contacting a cancer cell of the subject with a recombinant interleukin-27 (IL27) expressing virus. The recombinant IL27 expressing virus comprises an oncolytic virus comprising one or more exogenous nucleic acid sequences capable of expressing in IL27 protein or a biologically active portion thereof, the exogenous nucleic acid sequences being operably linked to an expression control sequence.

The recombinant IL27 expressing virus used in the method of treatment can include any of the oncolytic viruses described herein. In some embodiments, the oncolytic virus is a herpesvirus, while in further embodiments herpesvirus is an HSV-1 herpesvirus. In further embodiments, the oncolytic virus includes a deletion mutation that decreases the virulence of the oncolytic virus. In additional embodiments, the herpesvirus is modified to include a deletion of the herpesvirus gamma (1)34.5 gene (γ134.5). In a yet further embodiments, the IL27 protein comprises an amino acid sequence that is identical to the wild-type human IL27 sequence. In yet further embodiments, the nucleic acid sequences capable of expressing in IL27 protein are a bi-cistronic mIL27 gene inserted into the oncolytic herpesvirus at the γ134.5 locus.

The invention provides a method of treating cancer in a subject in need thereof using the recombinant IL27 expressing virus described herein. The term “cancer” refers to a proliferative disorder caused or characterized by a proliferation of cells which have lost susceptibility to normal growth control. Cancers of the same tissue type usually originate in the same tissue and may be divided into different subtypes based on their biological characteristics. Four general categories of cancer are carcinoma (epithelial cell derived), sarcoma (connective tissue or mesodermal derived), leukemia (blood-forming tissue derived) and lymphoma (lymph tissue derived).

Methods of treating cancer by in a subject by contacting a cancer cell of the subject with a recombinant IL27 expressing virus are described. The contacting step can be performed in vivo or ex vivo. Contacting the cancer cell involves putting the recombinant IL27 expressing virus in proximity to the motor neuron so that the IL27 expressed by the virus will interact with the cancer cell, or the immune system in the vicinity of the cancer cell. For example, the cancer cell can be contacted with the recombinant IL27 expressing virus by administering the recombinant IL27 expressing virus to a subject having cancer. The target cell can be a solid tumor cell. The disclosed recombinant IL27 expressing virus can also be used to treat a precancer condition such as cervical and anal dysplasia, other dysplasia, severe dysplasia, hyperplasia, atypical hyperplasia, or neoplasia. Thus, the target cancer cell can be a adenocarcinoma, hepatoblastoma, sarcoma, glioma, glioblastoma, neuroblastoma, plasmacytoma, histiocytoma, melanoma, adenoma, myeloma, bladder cancer, brain cancer, squamous cell carcinoma of the head and neck, ovarian cancer, skin cancer, liver cancer, lung cancer, colon cancer, cervical cancer, breast cancer, renal cancer, esophageal carcinoma, head and neck carcinoma, testicular cancer, colorectal cancer, prostatic cancer, or pancreatic cancer. The target cancer cells can be ectodermally-derived cancer cells. The target cancer cells can be brain cancer cells. Thus, the target cancer cell can be a neuroblastoma cell, glioma cell, or glioblastoma cell. In some embodiments, the cancer cell is glioblastoma. The target cancer cell can be a breast cancer cell. The target cancer cell can be a hepatoblastoma cell or liver cancer cell. The method of killing a targeted cancer cell can further comprise additional steps known in the art for promoting cell death.

An anti-viral immune response contributes to the effect of the recombinant IL27 expressing virus. The IL27 expressing virus induces interferon signaling, which recruits both the innate (e.g., neutrophils, NK cells, and macrophages) and adaptive (CD4+, CD8+) immune responses, as well as improved antigen recognition. The recombinant IL27 expressing virus also reverses the immunosuppressive tumor environment, and stimulates anti-tumor immune recognition.

Methods of treatment include administration of the recombinant IL27 expressing virus alone, or combination therapies wherein the animal is also undergoing one or more cancer therapies selected from the group consisting of surgery, chemotherapy, radiation therapy, thermotherapy, immunotherapy, hormone therapy and laser therapy.

In general any combination therapy will include one or more of chemotherapeutics, targeting agents like antibodies; kinase inhibitors; hormonal agents and the like. Combination therapies can also include conventional therapy, including, but not limited to, antibody administration, vaccine administration, administration of cytotoxic agents, natural amino acid polypeptides, nucleic acids, nucleotide analogues, and biologic response modifiers. Two or more combined compounds may be used together or sequentially. For example, anti-cancer agents that are well known in the art and can be used as a treatment in combination with the compositions described herein include, but are not limited to As used herein, a first line “chemotherapeutic agent” or first line chemotherapy is a medicament that may be used to treat cancer, and generally has the ability to kill cancerous cells directly.

Examples of chemotherapeutic agents include alkylating agents, antimetabolites, natural products, hormones and antagonists, and miscellaneous agents. Examples of alkylating agents include nitrogen mustards such as mechlorethamine, cyclophosphamide, ifosfamide, melphalan (L-sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU, 5FU), floxuridine (fluorodeoxyuridine, FUdR), cytarabine (cytosine arabinoside) and gemcitabine; purine analogs such as mercaptopurine (6-niercaptopurine, 6-MP), thioguanine (6-thioguanine, TG) and pentostatin (2′-deoxycoformycin, deoxycoformycin), cladribine and fludarabine; and topoisomerase inhibitors such as amsacrine. Examples of natural products include vinca alkaloids such as vinblastine (VLB) and vincristine; taxanes such as paclitaxel (Abraxane) and docetaxel (Taxotere); epipodophyllotoxins such as etoposide and teniposide; camptothecins such as topotecan and irinotecan; antibiotics such as dactinomycin (actinomycin D), daunorubicin (daunomycin, rubidomycin), doxorubicin, bleomycin, mitomycin (mitomycin C), idarubicin, epirubicin; enzymes such as L-asparaginase; and biological response modifiers such as interferon alpha and interlelukin 2. Examples of hormones and antagonists include luteinising releasing hormone agonists such as buserelin; adrenocorticosteroids such as prednisone and related preparations; progestins such as hydroxyprogesterone caproate, medroxyprogesterone acetate and megestrol acetate; estrogens such as diethylstilbestrol and ethinyl estradiol and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Examples of miscellaneous agents include thalidomide; platinum coordination complexes such as cisplatin (czs-DDP), oxaliplatin and carboplatin; anthracenediones such as mitoxantrone; substituted ureas such as hydroxyurea; methylhydrazine derivatives such as procarbazine (N-methylhydrazine, MIH); adrenocortical suppressants such as mitotane (o,p′-DDD) and aminoglutethimide; RXR agonists such as bexarotene; and tyrosine kinase inhibitors such as imatinib.

As used herein, the term “radiotherapeutic regimen” or “radiotherapy” refers to the administration of radiation to kill cancerous cells. Radiation interacts with various molecules within the cell, but the primary target, which results in cell death is the deoxyribonucleic acid (DNA). However, radiotherapy often also results in damage to the cellular and nuclear membranes and other organelles. DNA damage usually involves single and double strand breaks in the sugar-phosphate backbone. Furthermore, there can be cross-linking of DNA and proteins, which can disrupt cell function. Depending on the radiation type, the mechanism of DNA damage may vary as does the relative biologic effectiveness. For example, heavy particles (i.e. protons, neutrons) damage DNA directly and have a greater relative biologic effectiveness. Whereas, electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient). External beam radiation consists of X-rays and/or gamma rays, while brachytherapy employs radioactive nuclei that decay and emit alpha particles, or beta particles along with a gamma ray.

### Formulations and Methods of Administration

The recombinant IL27 expressing viruses and viral vectors described herein can be administered in vitro or in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Pharmaceutically acceptable carriers are well known in the art and include aqueous solutions such as physiologically buffered saline or other solvents or vehicles such as glycols, glycerol, vegetable oils (e.g., olive oil) or injectable organic esters. A pharmaceutically acceptable carrier can be used to administer the compositions of the invention to a cell in vitro or to a subject in vivo. A pharmaceutically acceptable carrier can contain a physiologically acceptable compound that acts, for example, to stabilize the composition or to increase the absorption of the agent. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. One skilled in the art would know that the choice of a pharmaceutically acceptable carrier, including a physiologically acceptable compound, depends, for example, on the route of administration of the polypeptide. For example, a physiologically acceptable compound such as aluminum monosterate or gelatin is particularly useful as a delaying agent, which prolongs the rate of absorption of a pharmaceutical composition administered to a subject. Further examples of carriers, stabilizers or adjutants can be found in Martin, Remington's Pharm. Sci., 15th Ed. (Mack Publ. Co., Easton, 1975), incorporated herein by reference.

Although certain routes of administration are provided in the foregoing description, according to the invention, any suitable route of administration of the vectors may be adapted, and therefore the routes of administration described above are not intended to be limiting. Routes of administration may including but are not limited to, intravenous, oral, buccal, intranasal, inhalation, topical application to a mucosal membrane or injection, including intratumoral, intradermal, intrathecal, intracisternal, intralesional or any other type of injection. Administration can be effected continuously or intermittently and will vary with the subject and the condition to be treated. One of skill in the art would readily appreciate that the various routes of administration described herein would allow for the inventive vectors or compositions to be delivered on, in, or near the tumor or targeted cancer cells. One of skill in the art would also readily appreciate that various routes of administration described herein will allow for the vectors and compositions described herein to be delivered to a region in the vicinity of the tumor or individual cells to be treated. “In the vicinity” can include any tissue or bodily fluid in the subject that is in sufficiently close proximity to the tumor or individual cancer cells such that at least a portion of the vectors or compositions administered to the subject reach their intended targets and exert their therapeutic effects.

Depending on the oncolytic virus and the cancer, the virus can be administered using a systemic or more targeted approach. Common forms of administration include intratumoral or intravenous administration. Intradermal injections of oncolytic virus are employed for melanoma, intracavitary injection for gliomas, and intraperitoneal injection for treatment of ovarian cancer are a few examples. The oncolytic viruses can be administered intravenously, intraperitoneally, intramuscularly, subcutaneously, intracavity, or transdermally. Thus, administration of the provided viruses and vectors to the brain can be intracranial, subdural, epidural, or intra-cisternal. For example, the provided viruses and vectors can be administered directly into the tumors by stereotactic delivery. In another aspect, the provided viruses and vectors are administered in liposomes, such as those known in the art or described herein. The provided viruses and vectors can be administered to cancers not in the brain intravascularly or by direct injection into the tumor.

Effective dosages and schedules for administering the compositions may be determined empirically, and making such determinations is within the skill in the art. For example, there are several brain tumor models that provide a mechanism for rapid screening and evaluation of potential toxicities and efficacies of experimental therapies. There are six separate human glioma xenograft models used for critical studies. Pandita et al., Genes Chromosomes Cancer 39(1): 29-36 (2004). There is also available a spontaneously derived syngeneic glioma model that does not express foreign antigens commonly associated with chemically or virally induced experimental tumors. Hellums et al., Neuro-oncol. 7(3): 213-24 (2005). Other animals models for a variety of cancers can be obtained, for example, from The Jackson Laboratory, 600 Main Street Bar Harbor, Me. 04609 USA, which provides hundreds of cancer mouse models. Both direct (histology) and functional measurements (survival) of tumor volume can be used to monitor response to oncolytic therapy. These methods involve the sacrifice of representative animals to evaluate the population, increasing the animal numbers necessary for the experiments. Measurement of luciferase activity in the tumor provides an alternative method to evaluate tumor volume without animal sacrifice and allowing longitudinal population-based analysis of therapy.

The dosage ranges for the administration of the compositions are those large enough to produce the desired effect in which the symptoms of the disease are affected. The dosage should not be so large as to cause adverse side effects, such as unwanted cross-reactions, anaphylactic reactions, and the like. The dosage can be adjusted by the individual physician in the event of any counterindications. Dosage can vary, and can be administered in one or more dose administrations daily, for one or several days.

The following example is included for purposes of illustration and is not intended to limit the scope of the invention.

### EXAMPLES

### Example 1: IL27-Expressing Oncolytic Virus

The inventors postulated that some of the tumor-promoting activities previously associated with IL27 were likely related to these shared subunits and that these other closely related cytokines were responsible for these effects rather than IL27. Alternatively, the suppressive effects may represent natural negative feedback mechanisms to reduce the immune response following IL27 expression (similar to what occurs following IL12 expression).

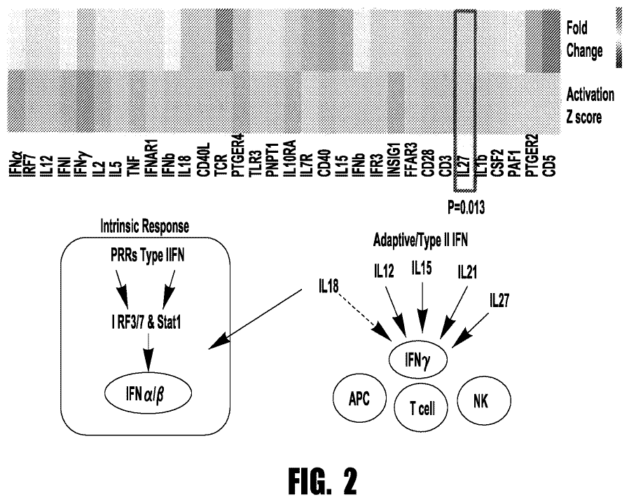

The inventors hypothesize that IL27 expressed from an oncolytic HSV would increase T cell and cytotoxic activity, reducing tumor growth, and improve survival in animals. Data supporting this hypothesis came from early clinical trial analysis, as shown in FIG. 2. When they assessed patients with improved survival after oHSV treatment, they identified genes associated with IL27 activity that directly correlated with improved patient survival.

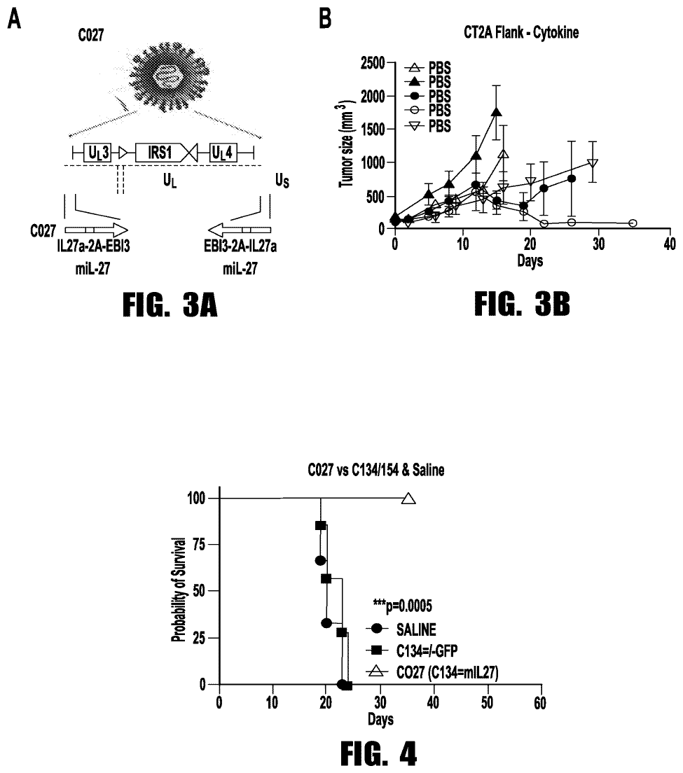

The inventors therefore created C027, an IL27-expressing oncolytic virus, to further test this hypothesis in immune component mouse models of cancer. As shown in FIG. 3, these studies confirmed their hypothesis that IL27 expression by oncolytic viruses improved survival and led to immune clearance of tumors.

One potential advantage of IL27 over the current IL12-based cytokine approach (that much of the field is using) is that IL27 exhibits both anti-tumor and anti-inflammatory activities. Current cytokine-based immunotherapies often produce dose limiting and dangerous cytotoxic side effects which limits their success. IL27 potentially may not exhibit this dose limiting toxicity although studies need to verify this. IL27 improves T cell Th1 function but it also has anti-inflammatory properties in other studies.

### Example 2: Peripheral Tumor Data

FIG. 4 provides data obtained in peripheral tumor tissue comparing the effects of C027, C134/154, and saline on probability of survival.

### Example 3: IL27 Expressing Virus Sequences

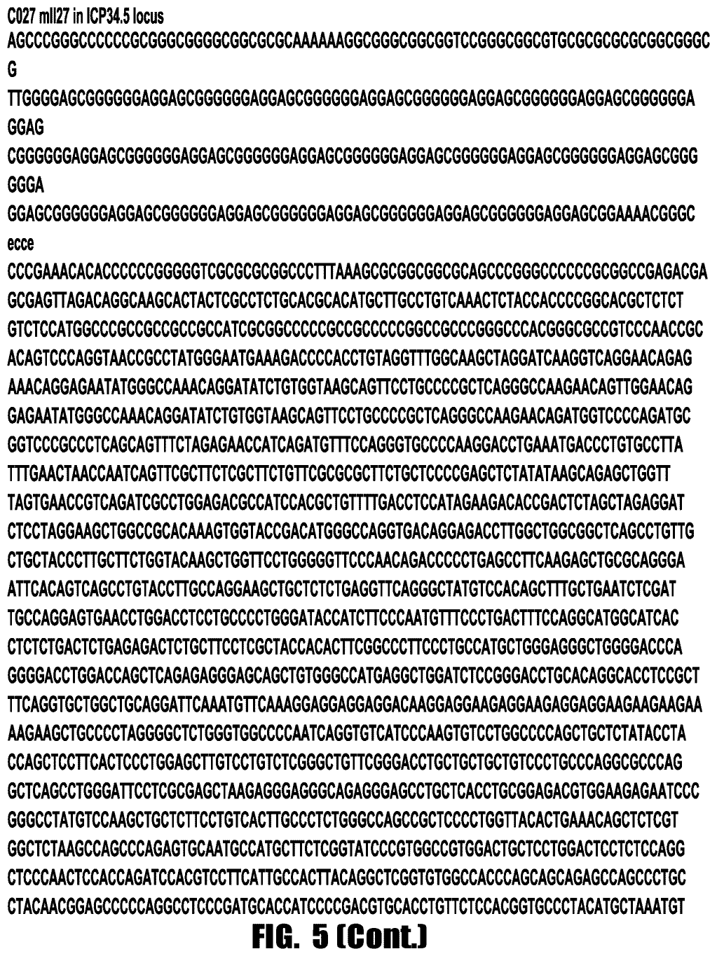

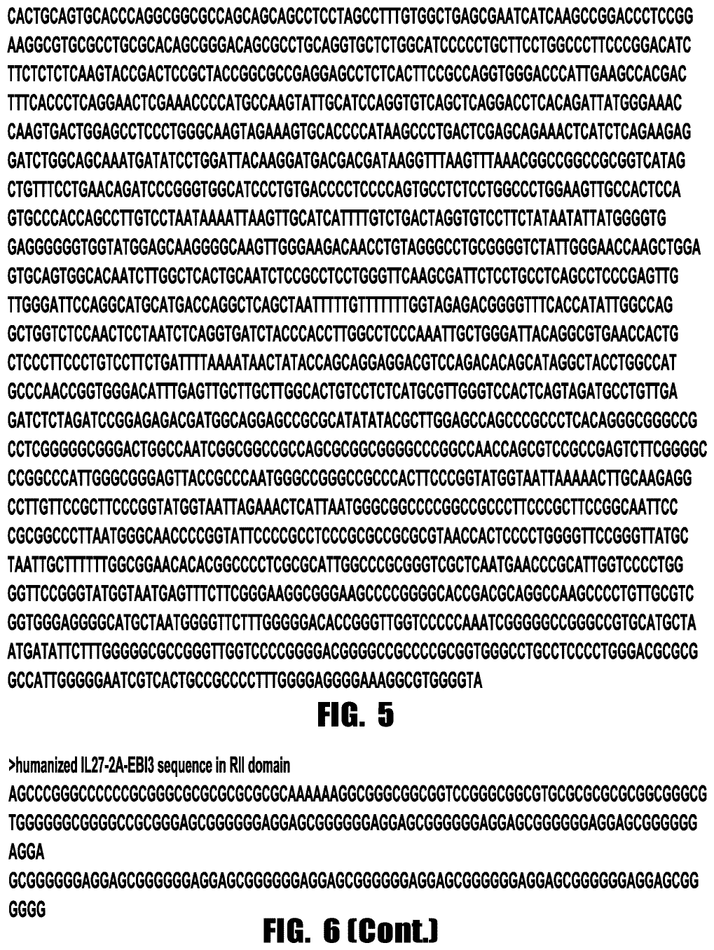

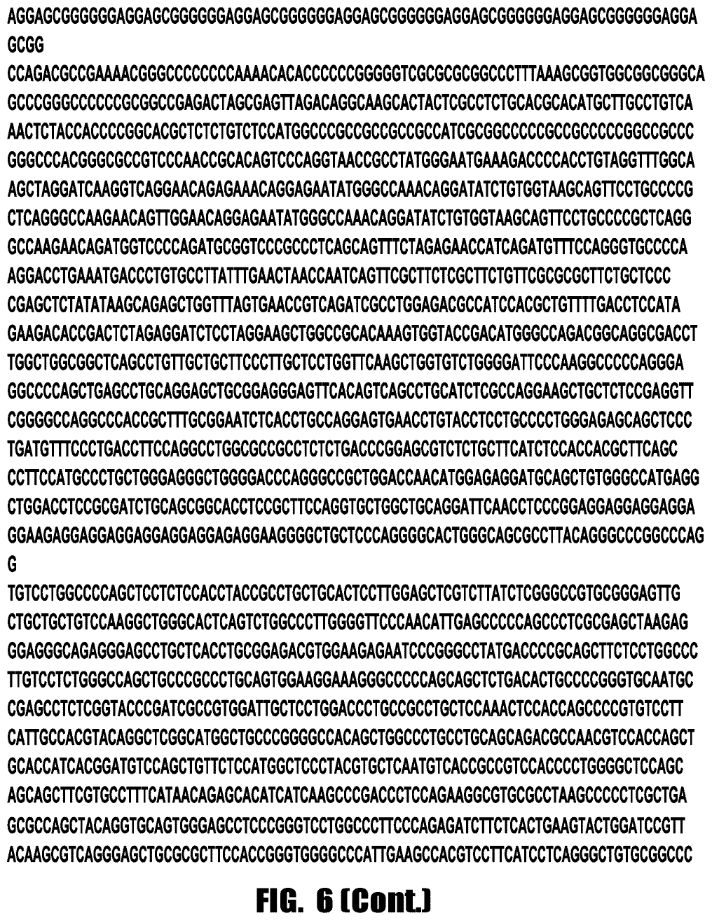

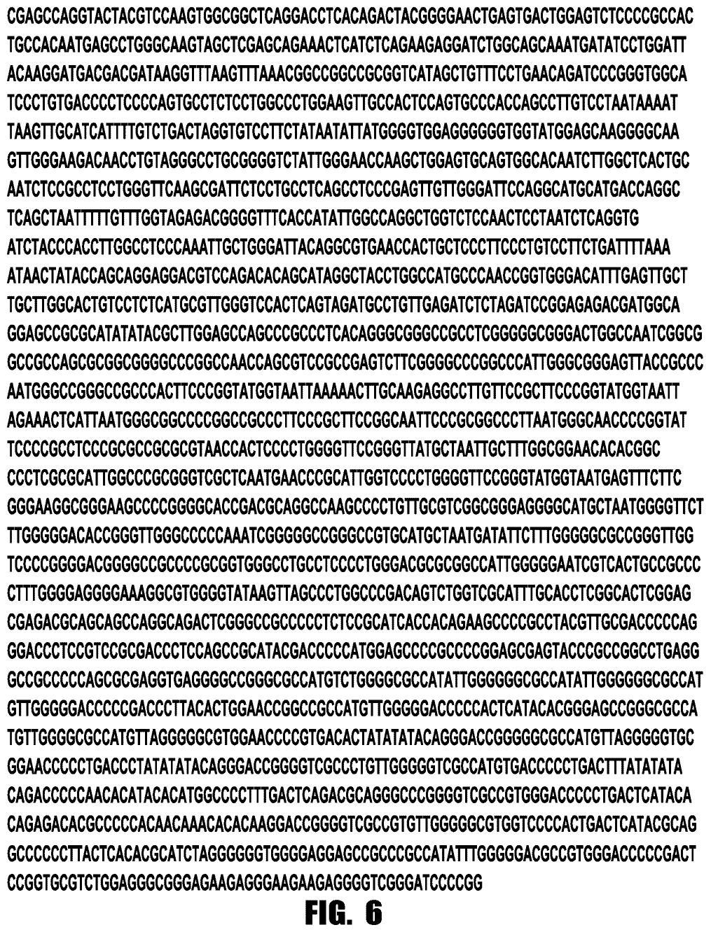

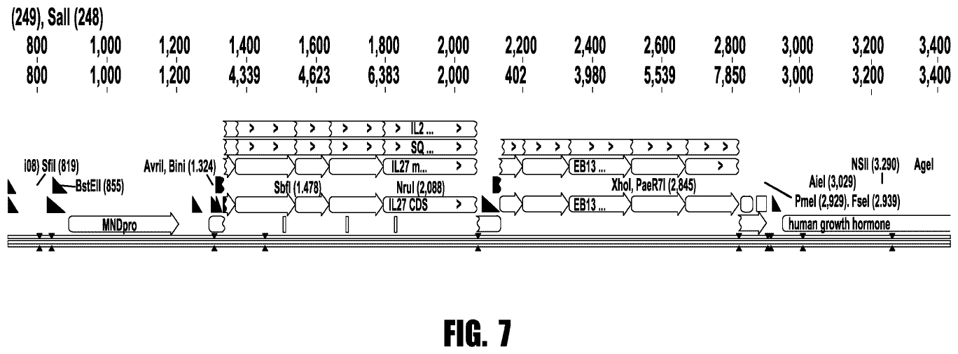

This example provides 1) a short (4056 NT) FastA sequence of the predicted domain in the C027 virus (FIG. 5), 2) the humanized IL27 RL1 viral sequence (FIGS. 6), and 3) humanized IL27 location in RL1 locus PNG image (FIG. 7).

The complete disclosure of all patents, patent applications, and publications, and electronically available materials cited herein are incorporated by reference. The foregoing detailed description and examples have been given for clarity of understanding only. No unnecessary limitations are to be understood therefrom. The invention is not limited to the exact details shown and described. for variations obvious to one skilled in the art will be included within the invention defined by the claims.

What is claimed is:

1. A recombinant interleukin-27 (IL27) expressing virus, comprising:
An oncolytic virus comprising one or more exogenous nucleic acid sequences capable of expressing in IL27 protein or a biologically active portion thereof, the exogenous nucleic acid sequences being operably linked to an expression control sequence.

2. The recombinant virus of claim 1, wherein the oncolytic virus includes a deletion mutation that decreases the virulence of the oncolytic virus.

3. The recombinant virus of claim 1, wherein the oncolytic virus is a herpesvirus.

4. The recombinant virus of claim 3, wherein the herpesvirus is an α herpesvirus.

5. The recombinant virus of claim 4, wherein the herpesvirus is an HSV-1 herpesvirus.

6. The recombinant virus of claim 2, wherein the virus is an HSV-1 herpesvirus, and modified to include a deletion of the herpesvirus gamma (1)34.5 gene (γ₁34.5) locus.

7. The recombinant virus of claim 6, further comprising a viral nucleic acid sequence encoding a PKR evasion protein that does not cause virulence.

8. The recombinant virus of claim 6, wherein the exogenous nucleic acid sequences operably linked to an expression control sequence and capable of expressing in IL27 protein or a functional portion thereof are inserted to replace the deleted γ₁34.5 locus.

9. The recombinant virus of claim 6, wherein the exogenous nucleic acid sequences capable of expressing in IL27 protein are a bi-cistronic mIL27 gene inserted into the oncolytic herpesvirus at the deleted γ₁34.5 locus.

10. The recombinant virus of claim 1, wherein the IL27 protein comprises an amino acid sequence that is at least 95% identical to the wild-type human IL27 sequence.

11. A method of treating cancer by in a subject by contacting a cancer cell of the subject with a recombinant interleukin-27 (IL27) expressing virus, the recombinant virus comprising:
An oncolytic virus comprising one or more exogenous nucleic acid sequences capable of expressing in IL27 protein or a biologically active portion thereof, the exogenous nucleic acid sequences being operably linked to an expression control sequence.

12. The method of claim 11, wherein the cancer is selected from the group consisting of adenocarcinoma, hepatoblastoma, sarcoma, glioma, glioblastoma, neuroblastoma, plasmacytoma, histiocytoma, melanoma, adenoma, myeloma, bladder cancer, brain cancer, squamous cell carcinoma of the head and neck, ovarian cancer, skin cancer, liver cancer, lung cancer, colon cancer, cervical cancer, breast cancer, renal cancer, esophageal carcinoma, head and neck carcinoma, testicular cancer, colorectal cancer, prostatic cancer, and pancreatic cancer cell.

13. The method of claim 11, wherein the cancer is glioblastoma.

14. The method of claim 11, wherein the cancer cell is contacted ex vivo.

15. The method of claim 11, wherein the cancer cell is contacted in vivo.

16. The method of claim 15, wherein the recombinant IL27 expressing virus is administered in a pharmaceutically acceptable carrier.

17. The method of claim 15, further comprising administering chemotherapy or radiation therapy to the subject.

18. The method of claim 11, wherein the oncolytic virus includes a deletion mutation that decreases the virulence of the oncolytic virus.

19. The method of claim 11, wherein the oncolytic virus is a herpesvirus.

20. The method of claim 19, wherein the herpesvirus is an HSV-1 herpesvirus.


SEQUENCE LISTING (3 sequences)

SEQ ID NO: 1 - DNA, 4055 bp, Homo sapiens
  source 1..4055: mol_type = other DNA
agcccgggccccccgcgggcggggcggcgcgcaaaaaaggcgggcggcggtccgggcggcgtgcgcgcgcgcggcgggcgttggggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggaaaacgggccccccccgaaacacaccccccgggggtcgcgcgcggccctttaaagcgcggcggcgcagcccgggccccccgcggccgagacgagcgagttagacaggcaagcactactcgcctctgcacgcacatgcttgcctgtcaaactctaccaccccggcacgctctctgtctccatggcccgccgccgccgccatcgcggcccccgccgcccccggccgcccgggcccacgggcgccgtcccaaccgcacagtcccaggtaaccgcctatgggaatgaaagaccccacctgtaggtttggcaagctaggatcaaggtcaggaacagagaaacaggagaatatgggccaaacaggatatctgtggtaagcagttcctgccccgctcagggccaagaacagttggaacaggagaatatgggccaaacaggatatctgtggtaagcagttcctgccccgctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctggtttagtgaaccgtcagatcgcctggagacgccatccacgctgttttgacctccatagaagacaccgactctagctagaggatctcctaggaagctggccgcacaaagtggtaccgacatgggccaggtgacaggagaccttggctggcggctcagcctgttgctgctacccttgcttctggtacaagctggttcctgggggttcccaacagaccccctgagccttcaagagctgcgcagggaattcacagtcagcctgtaccttgccaggaagctgctctctgaggttcagggctatgtccacagctttgctgaatctcgattgccaggagtgaacctggacctcctgcccctgggataccatcttcccaatgtttccctgactttccaggcatggcatcacctctctgactctgagagactctgcttcctcgctaccacacttcggcccttccctgccatgctgggagggctggggacccaggggacctggaccagctcagagagggagcagctgtgggccatgaggctggatctccgggacctgcacaggcacctccgctttcaggtgctggctgcaggattcaaatgttcaaaggaggaggaggacaaggaggaagaggaagaggaggaagaagaagaaaagaagctgcccctaggggctctgggtggccccaatcaggtgtcatcccaagtgtcctggccccagctgctctatacctaccagctccttcactccctggagcttgtcctgtctcgggctgttcgggacctgctgctgctgtccctgcccaggcgcccaggctcagcctgggattcctcgcgagctaagagggagggcagagggagcctgctcacctgcggagacgtggaagagaatcccgggcctatgtccaagctgctcttcctgtcacttgccctctgggccagccgctcccctggttacactgaaacagctctcgtggctctaagccagcccagagtgcaatgccatgcttctcggtatcccgtggccgtggactgctcctggactcctctccaggctcccaactccaccagatccacgtccttcattgccacttacaggctcggtgtggccacccagcagcagagccagccctgcctacaacggagcccccaggcctcccgatgcaccatccccgacgtgcacctgttctccacggtgccctacatgctaaatgtcactgcagtgcacccaggcggcgccagcagcagcctcctagcctttgtggctgagcgaatcatcaagccggaccctccggaaggcgtgcgcctgcgcacagcgggacagcgcctgcaggtgctctggcatccccctgcttcctggcccttcccggacatcttctctctcaagtaccgactccgctaccggcgccgaggagcctctcacttccgccaggtgggacccattgaagccacgactttcaccctcaggaactcgaaaccccatgccaagtattgcatccaggtgtcagctcaggacctcacagattatgggaaaccaagtgactggagcctccctgggcaagtagaaagtgcaccccataagccctgactcgagcagaaactcatctcagaagaggatctggcagcaaatgatatcctggattacaaggatgacgacgataaggtttaagtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgagatctctagatccggagagacgatggcaggagccgcgcatatatacgcttggagccagcccgccctcacagggcgggccgcctcgggggcgggactggccaatcggcggccgccagcgcggcggggcccggccaaccagcgtccgccgagtcttcggggcccggcccattgggcgggagttaccgcccaatgggccgggccgcccacttcccggtatggtaattaaaaacttgcaagaggccttgttccgcttcccggtatggtaattagaaactcattaatgggcggccccggccgcccttcccgcttccggcaattcccgcggcccttaatgggcaaccccggtattccccgcctcccgcgccgcgcgtaaccactcccctggggttccgggttatgctaattgcttttttggcggaacacacggcccctcgcgcattggcccgcgggtcgctcaatgaacccgcattggtcccctggggttccgggtatggtaatgagtttcttcgggaaggcgggaagccccggggcaccgacgcaggccaagcccctgttgcgtcggtgggaggggcatgctaatggggttctttgggggacaccgggttggtcccccaaatcgggggccgggccgtgcatgctaatgatattctttgggggcgccgggttggtccccggggacggggccgccccgcggtgggcctgcctcccctgggacgcgcggccattgggggaatcgtcactgccgcccctttggggaggggaaaggcgtggggta

SEQ ID NO: 2 - DNA, 4934 bp, Homo sapiens
  source 1..4934: mol_type = other DNA
agcccgggccccccgcgggcgcgcgcgcgcgcaaaaaaggcgggcggcggtccgggcggcgtgcgcgcgcgcggcgggcgtggggggcggggccgcgggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggccagacgccgaaaacgggccccccccaaaacacaccccccgggggtcgcgcgcggccctttaaagcggtggcggcgggcagcccgggccccccgcggccgagactagcgagttagacaggcaagcactactcgcctctgcacgcacatgcttgcctgtcaaactctaccaccccggcacgctctctgtctccatggcccgccgccgccgccatcgcggcccccgccgcccccggccgcccgggcccacgggcgccgtcccaaccgcacagtcccaggtaaccgcctatgggaatgaaagaccccacctgtaggtttggcaagctaggatcaaggtcaggaacagagaaacaggagaatatgggccaaacaggatatctgtggtaagcagttcctgccccgctcagggccaagaacagttggaacaggagaatatgggccaaacaggatatctgtggtaagcagttcctgccccgctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctggtttagtgaaccgtcagatcgcctggagacgccatccacgctgttttgacctccatagaagacaccgactctagaggatctcctaggaagctggccgcacaaagtggtaccgacatgggccagacggcaggcgaccttggctggcggctcagcctgttgctgcttcccttgctcctggttcaagctggtgtctggggattcccaaggcccccagggaggccccagctgagcctgcaggagctgcggagggagttcacagtcagcctgcatctcgccaggaagctgctctccgaggttcggggccaggcccaccgctttgcggaatctcacctgccaggagtgaacctgtacctcctgcccctgggagagcagctccctgatgtttccctgaccttccaggcctggcgccgcctctctgacccggagcgtctctgcttcatctccaccacgcttcagcccttccatgccctgctgggagggctggggacccagggccgctggaccaacatggagaggatgcagctgtgggccatgaggctggacctccgcgatctgcagcggcacctccgcttccaggtgctggctgcaggattcaacctcccggaggaggaggaggaggaagaggaggaggaggaggaggagaggaaggggctgctcccaggggcactgggcagcgccttacagggcccggcccaggtgtcctggccccagctcctctccacctaccgcctgctgcactccttggagctcgtcttatctcgggccgtgcgggagttgctgctgctgtccaaggctgggcactcagtctggcccttggggttcccaacattgagcccccagccctcgcgagctaagagggagggcagagggagcctgctcacctgcggagacgtggaagagaatcccgggcctatgaccccgcagcttctcctggcccttgtcctctgggccagctgcccgccctgcagtggaaggaaagggcccccagcagctctgacactgccccgggtgcaatgccgagcctctcggtacccgatcgccgtggattgctcctggaccctgccgcctgctccaaactccaccagccccgtgtccttcattgccacgtacaggctcggcatggctgcccggggccacagctggccctgcctgcagcagacgccaacgtccaccagctgcaccatcacggatgtccagctgttctccatggctccctacgtgctcaatgtcaccgccgtccacccctggggctccagcagcagcttcgtgcctttcataacagagcacatcatcaagcccgaccctccagaaggcgtgcgcctaagccccctcgctgagcgccagctacaggtgcagtgggagcctcccgggtcctggcccttcccagagatcttctcactgaagtactggatccgttacaagcgtcagggagctgcgcgcttccaccgggtggggcccattgaagccacgtccttcatcctcagggctgtgcggccccgagccaggtactacgtccaagtggcggctcaggacctcacagactacggggaactgagtgactggagtctccccgccactgccacaatgagcctgggcaagtagctcgagcagaaactcatctcagaagaggatctggcagcaaatgatatcctggattacaaggatgacgacgataaggtttaagtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgagatctctagatccggagagacgatggcaggagccgcgcatatatacgcttggagccagcccgccctcacagggcgggccgcctcgggggcgggactggccaatcggcggccgccagcgcggcggggcccggccaaccagcgtccgccgagtcttcggggcccggcccattgggcgggagttaccgcccaatgggccgggccgcccacttcccggtatggtaattaaaaacttgcaagaggccttgttccgcttcccggtatggtaattagaaactcattaatgggcggccccggccgcccttcccgcttccggcaattcccgcggcccttaatgggcaaccccggtattccccgcctcccgcgccgcgcgtaaccactcccctggggttccgggttatgctaattgcttttttggcggaacacacggcccctcgcgcattggcccgcgggtcgctcaatgaacccgcattggtcccctggggttccgggtatggtaatgagtttcttcgggaaggcgggaagccccggggcaccgacgcaggccaagcccctgttgcgtcggcgggaggggcatgctaatggggttctttgggggacaccgggttgggcccccaaatcgggggccgggccgtgcatgctaatgatattctttgggggcgccgggttggtccccggggacggggccgccccgcggtgggcctgcctcccctgggacgcgcggccattgggggaatcgtcactgccgcccctttggggaggggaaaggcgtggggtataagttagccctggcccgacagtctggtcgcatttgcacctcggcactcggagcgagacgcagcagccaggcagactcgggccgccccctctccgcatcaccacagaagccccgcctacgttgcgacccccagggaccctccgtccgcgaccctccagccgcatacgacccccatggagccccgccccggagcgagtacccgccggcctgagggccgcccccagcgcgaggtgaggggccgggcgccatgtctggggcgccatattggggggcgccatattggggggcgccatgttgggggacccccgacccttacactggaaccggccgccatgttgggggacccccactcatacacgggagccgggcgccatgttggggcgccatgttagggggcgtggaaccccgtgacactatatatacagggaccgggggcgccatgttagggggtgcggaaccccctgaccctatatatacagggaccggggtcgccctgttgggggtcgccatgtgaccccctgactttatatatacagacccccaacacatacacatggcccctttgactcagacgcagggcccggggtcgccgtgggaccccctgactcatacacagagacacgcccccacaacaaacacacaaggaccggggtcgccgtgttgggggcgtggtccccactgactcatacgcaggccccccttactcacacgcatctaggggggtggggaggagccgcccgccatatttgggggacgccgtgggacccccgactccggtgcgtctggagggcgggagaagagggaagaagaggggtcgggatccccgg

SEQ ID NO: 3 - protein, 243 aa, Homo sapiens
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP